(19) United States
(12) Patent Application Publication
(10) Pub. No.: US 2022/0096198 A1
(43) Pub. Date: Mar. 31, 2022

(54) DEVICES, SYSTEMS, AND METHODS FOR LOCATING A BODY LUMEN

(71) Applicant: Boston Scientific Scimed, Inc., Maple Grove, MN (US)

(72) Inventors: Ryan Desmond Lynch, Roscommon (IE); John Thomas Favreau, Spencer, MA (US); Marcia Nardone, Northborough, MA (US)

(21) Appl. No.: 17/477,990

(22) Filed: Sep. 17, 2021

Related U.S. Application Data
(60) Provisional application No. 63/084,033, filed on Sep. 28, 2020.

(51) Int. Cl. A61B 90/00

(52) U.S. Cl. CPC A61B 90/39; A61B 2090/3945; A61M 25/1018; A61B 2090/3912; A61B 2090/3962

(57) ABSTRACT

The present disclosure relates generally to systems and methods for performing endoscopic procedures, and, more particularly, to location and access devices, systems, and methods for gastrojejunostomy procedures. In an aspect a medical device locator may include an elongate member having a proximal end, a distal end, a longitudinal axis, and a length extending along the longitudinal axis. An inflatable member may be disposed about the distal end of the elongate member. The inflatable member may have a first inflated volume and a second inflated volume larger than the first inflated volume. The elongate member may include a fluid lumen extending therealong. The fluid lumen may be in fluid communication with the inflatable member and may be configured to pulsate the inflatable member between the first inflated volume and the second inflated volume.

### PRIORITY

The present application is a non-provisional of, and claims the benefit of priority under 35 U.S.C. § 119 to, U.S. Provisional Application Ser. No. 63/084,033, filed Sep. 28, 2020, the disclosure of which is hereby incorporated herein by reference in its entirety for all purposes.

### FIELD

The present disclosure relates generally to systems and methods for performing endoscopic procedures, and, more particularly, to location and access devices, systems, and methods for gastrojejunostomy procedures.

### BACKGROUND

Viewing, locating, and manipulating anatomies, devices, and/or anatomies containing one or more devices, such as a body lumen, may be difficult. During a procedure a medical professional may need to externally locate a particular portion of a body lumen. Locating a desired portion of a body lumen may be difficult due to a lack of or low amount of illumination, intervening anatomy, and/or lengthy additional portions of the body lumen. It is with respect to these and other considerations that the present improvements may be useful.

### SUMMARY

In one aspect of the present disclosure, a medical device locator may include an elongate member having a proximal end, a distal end, a longitudinal axis, and a length extending along the longitudinal axis. An inflatable member may be disposed about the distal end of the elongate member. The inflatable member may have a first inflated volume and a second inflated volume larger than the first inflated volume. The elongate member may include a fluid lumen extending therealong in fluid communication with the inflatable member. The fluid lumen may be configured to pulsate the inflatable member between the first inflated volume and the second inflated volume.

In various embodiments described herein or otherwise within the scope of the present disclosure, a first light emitting diode (LED) may be disposed along the inflatable member. The elongate member may be a multi-lumen catheter including a second lumen extending along the longitudinal axis of the catheter. The second lumen may include a lead electrically coupled to the first LED. The catheter may include a third lumen extending along the longitudinal axis of the catheter distally past the inflatable member, the third lumen configured to deliver a flow of contrast fluid. The first LED may be actuatable to emit a light having a wavelength corresponding to a green light, a red light, or both. The first LED may extend along the inflatable member substantially parallel with the longitudinal axis. A controller may be electrically communicative with the first LED. The controller may be configured to actuate the first LED in a pulsating fashion. The controller may be in communication with a reciprocating fluid supply. The controller may be configured to substantially synchronize the second inflated volume of the inflatable member with actuation of the first LED and substantially synchronize the first inflated volume of the inflatable member with a deactivation of the first LED. The controller may be in communication with the reciprocating fluid supply. The controller may be configured to transition the inflatable member between the first inflated volume and the second inflated volume at a first frequency. The controller may be configurated to actuate the first LED at a second frequency that is larger than the first frequency. A second LED may be disposed along the inflatable member and electrically communicative with the controller. The controller may be configured to sequentially actuate the first LED and the second LED. The first LED may extend helically about the longitudinal axis along a length of the inflatable member. The second inflated volume of the inflatable member may have a first diameter larger than a second diameter of a relaxed body lumen. The inflatable member in the first inflated volume may be extendable within a body lumen. The inflatable member in the second inflated volume may be configured to stretch a wall of the relaxed body lumen such that the wall of the body lumen about the inflatable member is thinner than the wall about the inflatable member in the first inflated volume. The inflatable member may be a compliant balloon. An oscillator may be coupled to the elongate member configured to oscillate the distal end of the elongate member across the longitudinal axis when actuated.

In an aspect, a system for locating a medical device may include a catheter extendable within a first body lumen. An inflatable member may be disposed about a distal end of the catheter in fluid communication with a fluid lumen extending along the catheter. The inflatable member may have a first inflated volume and a second inflated volume larger than the first inflated volume. An LED may be disposed along the inflatable member. An endoscope may be extendable within a second body lumen toward the catheter between opposing walls of the first and second body lumens.

In various embodiments described herein or otherwise within the scope of the present disclosure, a controller may be electrically coupled to a fluid supply in fluid communication with the inflatable member. The controller may be configured to actuate the inflatable member between the first inflated volume and the second inflated volume. The controller may be electrically coupled to the LED and may be configured to substantially synchronize the second inflated volume of the inflatable member with actuation of the LED and substantially synchronize the first inflated volume of the inflatable member with deactivation of the LED.

In an aspect, a method of locating a medical device across a wall of a first body lumen may include extending an inflatable member comprising an LED within the first body lumen. The inflatable member may pulsate between a first inflated volume and a second inflated volume. The LED may be actuated during the pulsated second inflated volume. The actuated LED, the second inflated volume of the inflatable member, or both, may be used to locate the medical device from across the wall of the first body lumen.

In various embodiments described herein or otherwise within the scope of the present disclosure, a cutting tool from within a second body lumen, may be extended across a wall of the second body lumen toward the wall of the first body lumen and cutting into the wall of the first body lumen. Viewing actuated LED may be from within the peritoneal cavity space between the wall of the first body lumen and the wall of the second body lumen.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 illustrates a gastrointestinal system of a patient.

FIG. 2A illustrates a medical device locator extending into the jejunum and a cutting tool cutting through a wall of the stomach, according to an embodiment of the present disclosure.

FIG. 2B illustrates the medical device locator of FIG. 2A in an inflated volume configuration and a grasping tool extending through the stomach wall towards a desired portion of the jejunum.

FIG. 2C illustrates the medical device locator of FIGS. 2A and 2B with the grasping tool manipulating a desired portion of the jejunum toward the stomach.

FIG. 2D illustrates the medical device locator of FIGS. 2A-2C with a cutting tool cutting a desired portion of the jejunum.

FIG. 3A illustrates a medical device locator in a first inflated volume configuration extending within a body lumen, according to an embodiment of the present disclosure.

FIG. 3B illustrates the medical device locator of FIG. 3A in a second inflated volume configuration.

FIG. 4 illustrates a medical device locator extending into the jejunum and an endoscope extending through the wall of the stomach toward the medical device locator, according to an embodiment of the present disclosure.

FIG. 5 illustrates a medical device locator having an injection needle extending within a body lumen, according to an embodiment of the present disclosure.

FIG. 6 illustrates a medical device locator having an array of injection needles, according to an embodiment of the present disclosure.

### DETAILED DESCRIPTION

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure may be described with specific reference to medical devices and systems (e.g., endoscopic devices, accessory tools, and/or guidewires inserted near or through a jejunum, or the like), it should be appreciated that such medical devices and systems may be used in a variety of medical procedures for navigating one or more devices through ductal, luminal, vascular, or body lumen anatomies, including, for example, interventional radiology procedures, balloon angioplasty/angiography procedures, thrombolysis procedures, urological or gynecological procedures, and the like. The medical devices herein may include a variety of medical devices for navigating body lumens, including, for example, catheters, ureteroscopes, bronchoscopes, colonoscopes, arthro scopes, cystoscopes, hysteroscopes, and the like. The disclosed medical devices and systems may also be inserted via different access points and approaches, e.g., percutaneously, endoscopically, laparoscopically, or combinations thereof.

As used herein, the singular forms “a,” “an,” and “the” are intended to include the plural forms as well, unless the context clearly indicates otherwise. The terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, “proximal end” refers to the end of a device that lies closest to the medical professional along the device when introducing the device into a patient, and “distal end” refers to the end of a device or object that lies furthest from the medical professional along the device during implantation, positioning, or delivery.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g., 1 to 5 includes 1, 1.5, 2, 2.75, 3, 3.80, 4, and 5).

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art. Exemplary devices, systems, and methods with which embodiments of the present disclosure may be implemented include, but are not limited to, those described in the complete disclosures of U.S. Pub. No. 2018/0271530, filed Mar. 26, 2018, published Sep. 27, 2018, and titled, “Systems and Methods to Effect Movement of Tissue Structures,” International Pub. No. 2019/140097, filed Jan. 10, 2019, published Jul. 18, 2019, and titled, “Systems, Methods And Devices For Connecting Non-Adherent Structures,” and U.S. Provisional Patent Application having an Attorney Docket No. 8150.0739Z, filed Sep. 1, 2020 and titled, “Grasping Devices, Systems, and Methods for Tissue Approximation and Apposition,” each of which disclosures are herein incorporated by reference in their entirety.

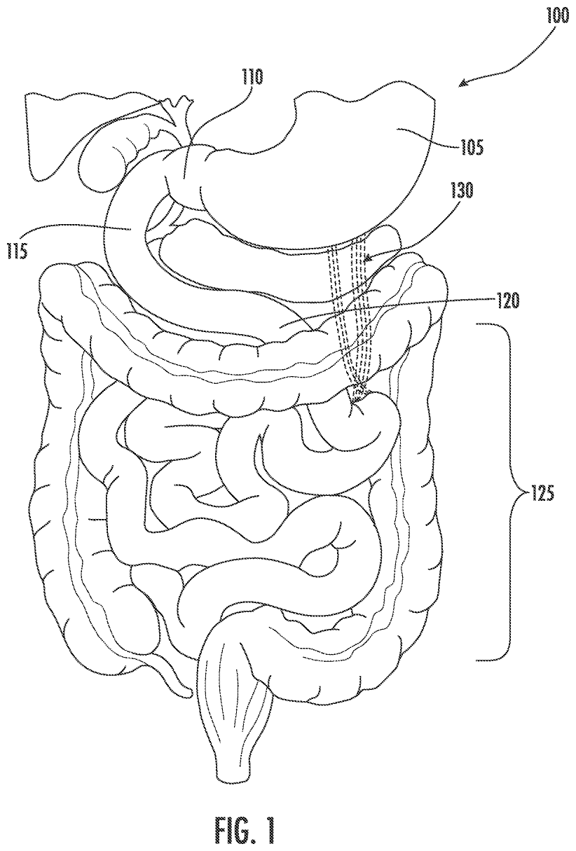

FIG. 1 illustrates a gastrointestinal system of a patient. According to exemplary embodiments of the present disclosure, a natural orifice transluminal endoscopic surgery (NOTES) procedure may be advantageous over other types of bypass procedures from a stomach 105 to a jejunum 120 (e.g., an endoscopic ultrasound procedure) so that a jejunal loop, or a loop of small bowel in the jejunum, may be selected a distance from the pylorus 110, for example, by creating an anastomosis farther into the jejunum 120 that may not otherwise be possible using other systems. In this manner, absorption of stomach contents (e.g., food and other nutrients) in the duodenal portion may be bypassed and uptake of nutrients from such contents may not be absorbed, or absorption may delayed, as such contents travel from the stomach 105 through the small bowel 125, promoting patient weight loss and possible controlling or resolving type-2 diabetes. For example, conditions of a disease may be benefitted by bypassing a portion of the jejunum 120, e.g., creating an anastomosis, e.g., about 150 cm or greater from the pylorus 110 with the duodenum 115.

In many embodiments, the present disclosure relates to devices, systems, and methods for performing a procedure, e.g., an endoscopic, laparoscopic, and/or open surgical procedure, to create a gastrojejunal anastomosis. For example, devices and systems described herein may aide gastrojejunal anastomosis placement by reliably and repeatably locating a desired position in a patient's gastrointestinal system, e.g., distinguishing a position in the jejunum 120 proximal and/or distal to the Ligament of Treitz 130. Additionally, devices and systems herein may allow for a medical professional to locate, grasp, hold, and/or cut a portion of the small bowel 125 during a gastrojejunal anastomosis procedure. Optionally a stent or other conduit may be placed across the bypass bridging the walls of the stomach and jejunum where the openings are created. The stent or conduit may assist with establishing or maintaining the anastomosis open until it is stable. The stent may or may not be subsequently removed. Although the systems and devices are described herein with respect to a gastrointestinal system, it may be understood that exemplary embodiments of devices and systems in accordance with the present disclosure may be advantageous for use in any other procedures and/or anatomy, where selective location is blind and grasping, manipulation, or cutting of tissue (e.g., a body lumen and/or other sensitive tissue structures) is indicated.

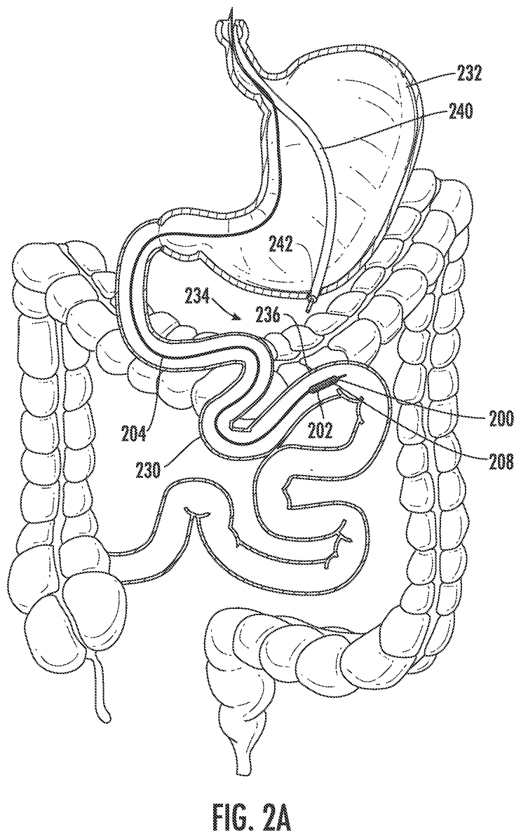

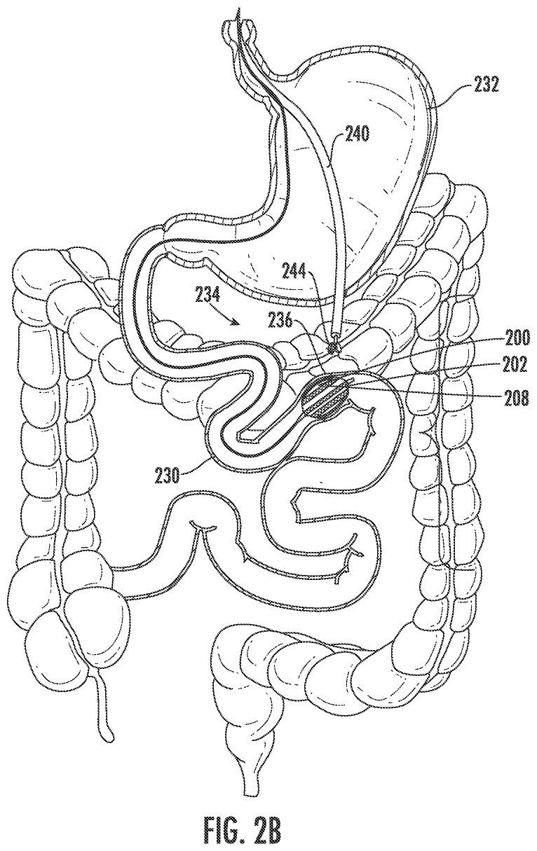

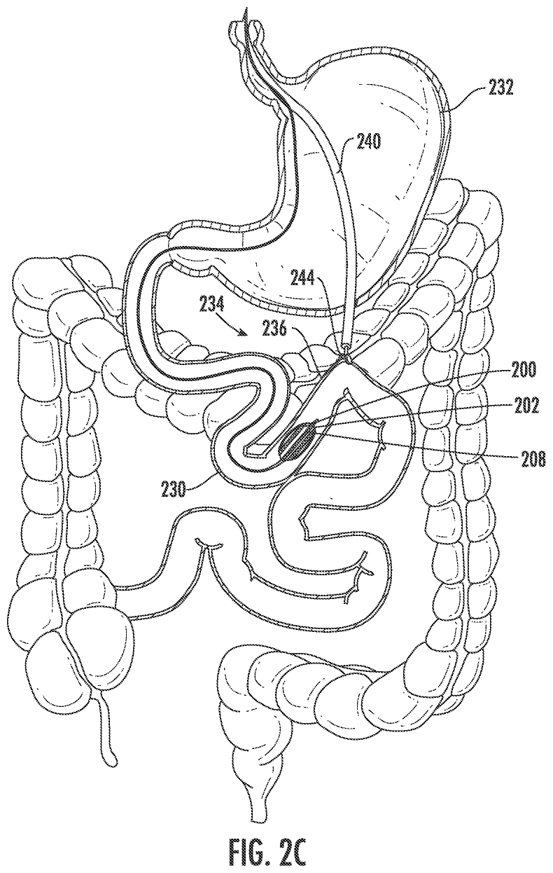

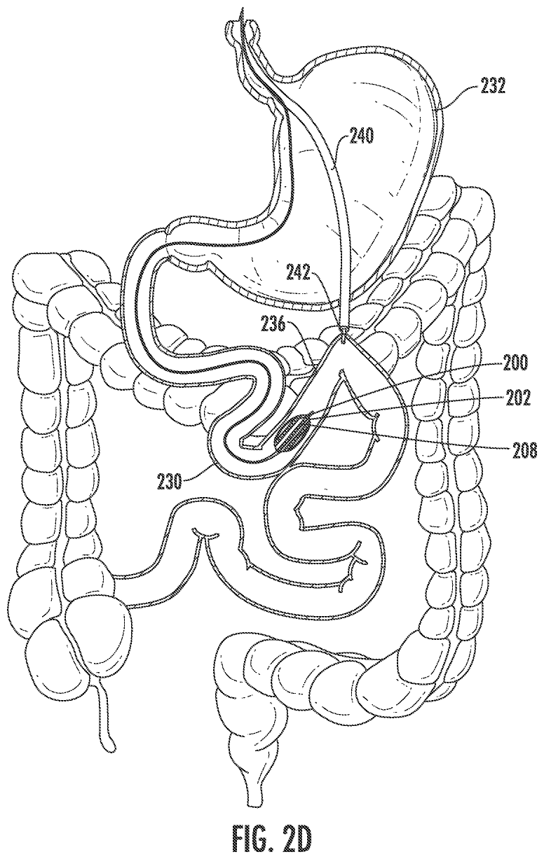

Referring to FIG. 2A, an embodiment of a medical device locator 202 is illustrated extending into the jejunum 230. The device locator 202 includes an elongate member 204 extending through the esophagus or nose to the stomach 232 and into the duodenum and jejunum 230. A distal end of the elongate member 204 includes an inflatable member 200. In various embodiments described herein, an inflatable member may comprise a balloon, a membrane, an expandable member such as a mesh or a braid, or a layered combination thereof. The inflatable member 200 is partially inflated at a first inflation volume. The inflatable member 200 includes multiple light emitting diodes (LEDs) 208 disposed along the inflatable member 200. In various embodiments described herein, an LED may include a flexible circuit. The LED and/or the flexible circuit may be disposed along a member by, e.g., an adhesive that may be partially or selectively applied to the LED and/or the flexible circuit, or the LED and/or flexible circuit may be formed partially or wholly within the inflatable member, e.g., allowing expansion/inflation and contraction/deflation of the inflatable member with the LEDs. The inflatable member 200 is positioned within a desired portion 236 of the jejunum 230, e.g., about 150 cm from the pylorus. Positioning of the inflatable member 200 may be assisted by peristaltic migration through the GI and/or axial translation of the elongate member 204. An endoscope 240 is extending through the esophagus into the stomach 232. A distal end of the endoscope 240 is positioned toward a wall of the stomach 232. A cutting tool 242 is extending from the distal end of the endoscope 240 such that it is cutting through the wall of the stomach 232 to access the peritoneal cavity 234 between the stomach wall and jejunum at the location of the medical device locator.

Referring to FIG. 2B, the medical device locator 202 of FIG. 2A is illustrated in an inflated configuration at a second inflation volume larger than the first inflation volume of FIG. 2A. The inflatable member 200 is in contact with the tissue wall of the jejunum 230 at the desired portion 236. The inflatable member 200 in the second inflated volume has a larger outer diameter than the inner diameter of the jejunum 230. The second inflated volume of the inflatable member 200 causes the desired portion 236 of the jejunum 230 to expand radially outwards such that an outer diameter of the desired portion 236 is larger than adjacent portions of the jejunum 230. Additionally, the LEDs 208 of the expandable member 200 may be actuated such that the desired portion 236 is illuminated. The distal end of the endoscope 240 is extending through the cut wall of the stomach 232 into the peritoneal cavity 234. The distal end of the endoscope 240 is oriented in a direction substantially toward the jejunum 230 within the peritoneal cavity 234. The medical professional may view the location of the desired portion 236 of the jejunum 230 because of the larger outer diameter of the desired portion 236 produced by the inflated inflatable member 200 compared to the outer diameter of adjacent portions of the jejunum 230. The medical professional may also or instead view the location of the desired portion 236 of the jejunum 230 because of the illuminated desired portion 236 produced by the LEDs 208 compared to the adjacent portions of the jejunum 230. A grasping tool 244 is extended through the endoscope 240 toward the located desired portion 236.

Referring to FIG. 2C, the medical device locator 202 of FIGS. 2A and 2B is illustrated with the grasping tool 244 grasping the desired portion 236 of the jejunum 230. Because the desired portion 236 is located and grasped by the grasping tool 244, the inflatable member 200 may be proximally withdrawn within the jejunum away from the desired portion 236 for the procedure to continue operation on the desired portion 236 and/or the inflatable member 200 may be at least partially uninflated from the second inflated volume such as to avoid collision with other medical tools or devices. However, the inflatable member 200 may instead remain substantially in the same position and/or at the same inflated volume during the procedure. The grasping tool 244 is manipulating the desired portion 236 of the jejunum such that the desired portion 236 is moved towards the stomach 232. Such positioning of the desired portion 236 may be performed by proximally translating the endoscope 240 and/or the grasping tool 244 while grasping the desired portion 236.

Referring to FIG. 2D, the medical device locator 202 of FIGS. 2A-2C is illustrated with the desired portion 236 of the jejunum 230 in the position moved toward the wall opening in stomach 232 as grasped and moved by the grasping tool in FIG. 2C. The cutting tool 242 is extended through the endoscope 240 to cut through the wall of the desired portion 236 of the jejunum 230. Wall openings of the stomach 232 and/or the jejunum 230 may then be apposed and fixed with respect to each other in positions to prevent fluid leakage into the peritoneal cavity. A tubular stent that may include flanges may be delivered to the openings and extend across the openings to bridge the walls of the stomach 232 and the jejunum 236 to assist with establishing or maintaining anastomosis and patency of the wall openings.

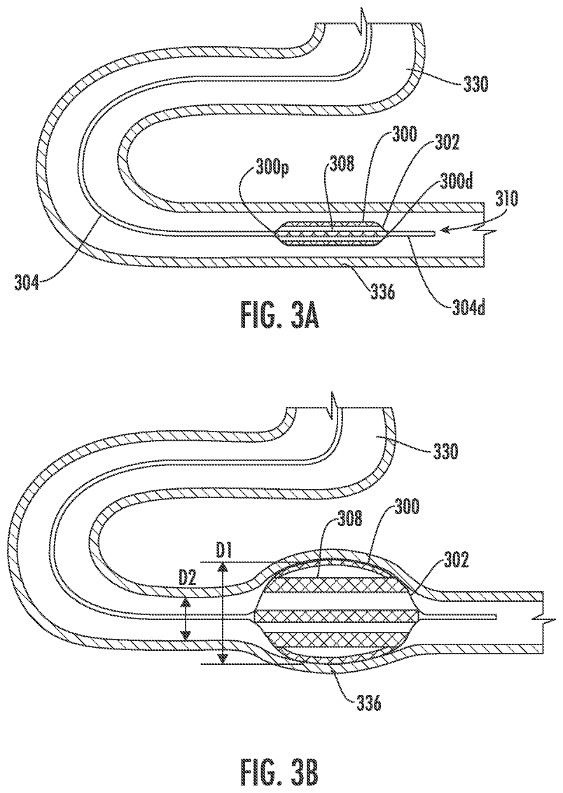

Referring to FIG. 3A, an embodiment of a medical device locator 302 is extended through a body lumen 330. The device locator 302 includes an elongate member 304 having a proximal end, a distal end 304d, a length extending along a longitudinal axis, and a first lumen 310 therethrough. The first lumen 310 may be configured to accommodate a tool, e.g., a guidewire or the like, and/or a fluid, e.g., a contrast fluid or the like. An inflatable member 300 is disposed about the distal end 304d of the elongate member 304 and is locatable at a desired portion 336 of the body lumen 330. The inflatable member 300 is partially inflated to a first inflated volume. The inflated volume of the inflatable member 300 may be adjusted by supplying and/or returning a fluid through a fluid lumen extending along the elongate member 304 in fluid communication with the inflatable member 300. The inflatable member 300 includes a proximally reducing tapered proximal end 300p and a distally reducing tapered distal end 300d that may reduce trauma and/or friction when translating the inflatable member 300 through the body lumen 330. The inflatable member 300 includes one or a multiple of LEDs 308 disposed along the inflatable member 300. Although the LEDs 308 are illustrated substantially parallel with a longitudinal axis of the inflatable member 300, any configuration of the LEDs 308 may be employed, e.g., helical, radial rings, symmetrical arrays about the inflatable member 300, asymmetrical arrays about the inflatable member 300, stripes, dots, a combination thereof, or the like. In the first inflated volume configuration, the inflatable member 300 may be more easily translated through the body lumen 330 than in a deflated configuration because the inflatable member 300 may be more rigid in the first inflated volume configuration improving pushability through the body lumen 330 compared to the more flexible deflated member 300. The inflatable member 300 may be more easily translated through the body lumen 330 in the first inflated volume configuration compared to a second, larger inflated volume configuration because there may be less friction between the inflatable member 300 and walls of the body lumen 330.

Referring to FIG. 3B, the medical device locator 302 of FIG. 3A is illustrated having a second inflated volume larger than the first inflated volume of FIG. 3A. The second inflated volume of the inflatable member 300 enlarges the inflatable member 300 such that it has a diameter D₁ larger than the internal diameter D₂ of the body lumen 330. The inflatable member 300 is contacting the walls of the desired portion 336 of the body lumen 330 such that the desired portion 336 expands to be larger in diameter than adjacent portions of the body lumen 330. Additionally, the LEDs 308 are brought into proximity or into contact with the walls of the desired portion 336 such that when the LEDs 308 are actuated, illumination of the LEDs may be externally viewable through the tissue wall of the desired portion 336.

In various embodiments, an inflatable member may pulsate by repeatedly transitioning between a first inflation volume and a larger second inflation volume. When positioned within a desired portion of a body lumen, the pulsating repeated transitioning of the inflatable volume between the first inflated volume and the second inflated volume may temporarily and repeatably expand the desired portion of the body lumen to be larger than adjacent portions of the body lumen. Transfer of such pulsating volume variances from the inflatable member to the desired portion of a body lumen may assist with identifying the desired portion when viewed externally from the body lumen.

In various embodiments, LEDs along an inflatable member may be actuated such that they are viewable across a tissue of a desired portion of a body lumen. An inflated inflatable member may bring the LEDs into contact with tissue of the body lumen. The larger diameter of an inflatable member compared to the inner diameter of the body lumen may cause circumferential apposition to stretch out the elastic tissue of the body lumen at the desired portion such that the tissue is thinner at the desired portion than at adjacent portions and light emitted from the LEDs more easily passes through the thinned tissue of the desired portion.

In various embodiments, a controller may control inflation fluid and/or actuation of LEDs of an inflation member via a multi-lumen elongate member including one or more fluid lumens and one or more electrical leads. Actuation of the LEDs may be synchronized with a second larger inflation volume of an inflatable member such that the LEDs are only actuated when they are more likely viewable in proximity to the tissue wall of a body lumen compared to a remote position away from the tissue. The LEDs may be synchronized such that they are deactivated during a first smaller inflation volume of an inflatable member. LED synchronization may reduce heat and/or energy consumption of a device. Synchronization actuation of the LEDs with a second larger inflation volume of the inflatable member may be at different frequencies. For example, the frequency of actuating the LEDs may be larger (i.e., shorter duration intervals) than the frequency of the second inflation volume such that the LEDs may illuminate multiple times during a single duration of the larger second inflation volume, which may be more noticeable to a medical professional viewing a desired portion of a body lumen from external to the body lumen, compared to a smaller actuation frequency of the LEDs (i.e., longer duration intervals). One or more LEDs may have variable illumination wavelengths that correspond to visual colors, e.g., red, green, yellow, blue, white, a combination thereof, or the like that may be more viewable to a medical professional than other colors. Multiple LEDs may be sequentially actuated.

In various embodiments, an inflatable member and/or an elongate member may include a vibrating or oscillating body. The vibrating or oscillating body may be actuated to move the inflatable member such that motion is transferred to a desired portion of a body lumen for external visual locating. Alternatively, a vibrating or oscillating body may replace an inflatable member of an embodiment herein such that the elongate member transfers motion to a desired portion of a body lumen for external visual locating.

In various embodiments, an inflatable member may comprise a compliant material. A compliant material may allow for the inflatable member to conform to walls of a desired portion of a body lumen when inflated. An inflatable member may comprise a non-compliant material. A non-compliant material may allow for a particular shape of the inflatable member and may not stress the LEDs of the inflatable member compared to a compliant material.

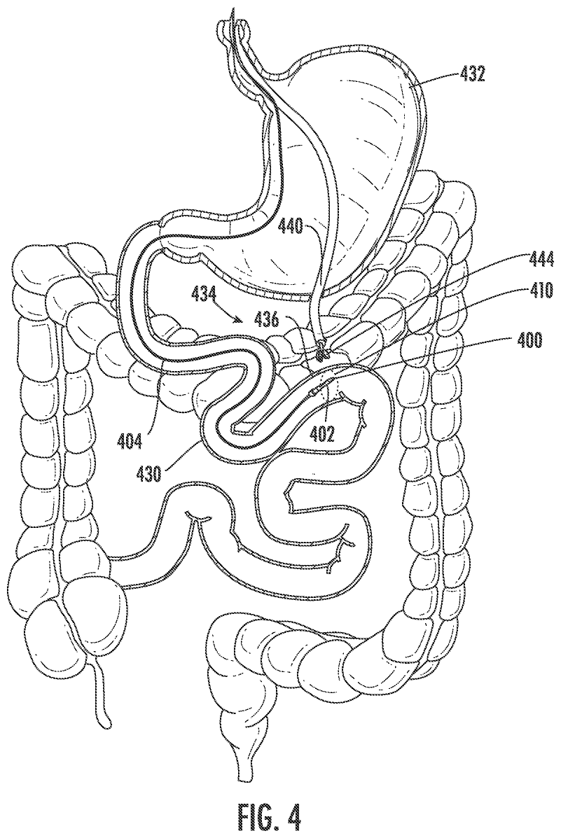

Referring to FIG. 4, an embodiment of a medical device locator 402 extending into the jejunum 430 and marking a desired portion 436 of the jejunum 430 is illustrated. The device locator 402 includes an elongate member 404 extending through the esophagus, stomach 432, duodenum, and to the desired portion 436 of the jejunum 430. The elongate member 404 may include a sheath that may be axially translated with respect to a fluid needle 400 at a distal end of the elongate member 404 to reveal the fluid needle 400. The fluid needle 400 may be used to penetrate and inject a fluid 410 into tissue of the desired portion 436. The fluid 410 may be visible external to the jejunum 430 from an endoscope 440 extending into the peritoneal cavity 434 (via methods described herein, e.g., as illustrated and described with respect to FIGS. 2A-2D). Although the fluid 410 is dispersed at the desired portion 436 in a dotted ring-like shape, any shape may additionally or alternatively be employed, e.g., one or more segments such as four quadrant injections around the circumference of the jejunum 430.

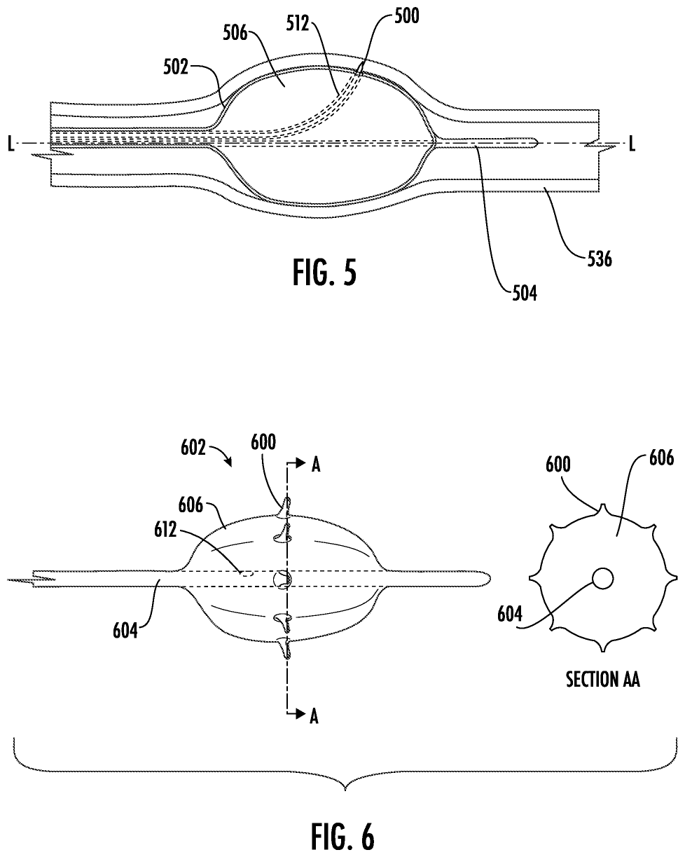

Referring to FIG. 5, an embodiment of a medical device locator 502 including an expandable member 506 is illustrated extending within a body lumen 536. The medical device locator 502 may be deliverable to the body lumen 536 by translating an elongate member 504 extending through the medical device locator. The expandable member 506 is expanded against the wall of the body lumen 536. The medical device locator 502 includes a needle lumen 512 extending through the expandable member 506 at an angle away from a longitudinal axis of the medical device locator 502. A needle 500 is slidably disposed through the needle lumen 512. The needle 500 is illustrated extending distally out of the needle lumen 512 into the wall of the body lumen 536 to inject a fluid, e.g., a fluid dye for marking the body lumen 536 as described herein. For example, the needle 500 may be extended into a submucosa where a bleb may be formed by supplying about 0.5 mL to about 1.0 mL to about 2.0 mL of fluid to confirm the needle 500 is not transmural across the tissue wall. The needle 500 may be proximally and distally extended through the needle lumen 512, e.g., to withdraw or deploy the needle 500 with respect to the expandable member 506. The needle 500 may mark multiple portions of the wall of the body lumen 536, e.g., in a ring-like pattern, by proximally withdrawing the needle 500 into the needle lumen 512 and manipulating the medical device locator 502 such that a distal end of the needle lumen 512 is oriented at another portion of the wall of the body lumen 536. This manipulation of the medical device locator 502 may be, e.g., rotating the medical device locator 502 about the longitudinal axis and/or extending the medical device locator 502 longitudinally along the longitudinal axis. Once the medical device locator 502 is manipulated, the needle 500 may be distally extended through the needle lumen 512 to mark another portion of the wall of the body lumen 536.

Referring to FIG. 6, an embodiment of a medical device locator 602 is illustrated having an expandable member 606. The medical device locator 602 may be manipulated by translating an elongate member 604 extending through the medical device locator. The medical device locator 602 includes multiple needles 600 extending from and arrayed about the expandable member 606. The expandable member 606 is in an expanded configuration with the needles extending away from the elongate member 604, e.g., for engaging tissue. The expandable member 606 may be expanded, e.g., by supplying fluid through an aperture 612 along the elongate member 604 and into the expandable member 606. The fluid may be a fluid dye for marking tissue as described herein, which may flow from the aperture 612, into the expandable member 606, through the needles 600, and into tissue. Fluid may be withdrawn through the aperture 612 and the elongate member 604 to unexpand the expandable member 606 and to disengage the needles 600 from tissue for withdrawal or relocation of the medical device locator 602 to another portion of the body, e.g., for further marking.

In various embodiments, a fluid from a fluid needle may be a visible medical ink. A fluid needle may be configured to deliver a fluid into layers of a tissue wall of a body lumen. A fluid may be a desirable medium for marking a tissue because it does not introduce significant heat into the body even during prolonged exposure, compared to other electro-mechanical methods of locating a desired portion of a body lumen that may dissipate heat. A fluid may comprise a fluorescent ink that illuminates, i.e., glows, when succumbed to particular light conditions, e.g., about 465 nm wavelength light or the like. A fluid may comprise various fluorescent materials such as fluorescent supramolecular nanoparticles encapsulating a fluorescent conjugated polymer, or the like. Various light wavelengths may be delivered through an endoscope, e.g., via fiber optics or from a light source within the endoscope itself. Fluids visible under various lighting conditions may differentiate marked anatomies. A fluid may comprise a biodegradable material.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A medical device locator comprising:
an elongate member having a proximal end, a distal end, a longitudinal axis and a length extending along the longitudinal axis;
an inflatable member disposed about the distal end of the elongate member, the inflatable member having a first inflated volume and a second inflated volume larger than the first inflated volume; and
wherein the elongate member comprises a fluid lumen extending therealong the fluid lumen in fluid communication with the inflatable member, and the fluid lumen configured to pulsate the inflatable member between the first inflated volume and the second inflated volume.

2. The medical device locator of claim 11, further comprising a first light emitting diode (LED) disposed along the inflatable member.

3. The medical device locator of claim 1, wherein the elongate member is a multilumen catheter including a second lumen extending along the longitudinal axis of the catheter, the second lumen comprising a lead electrically coupled to the first LED.

4. The medical device locator of claim 3, wherein the catheter further comprises a third lumen extending along the longitudinal axis of the catheter distally past the inflatable member, the third lumen configured to deliver a flow of contrast fluid.

5. The medical device locator of claim 2, wherein the first LED is actuatable to emit a light having a wavelength corresponding to a green light, a red light, or both.

6. The medical device locator of claim 2, wherein the first LED extends along the inflatable member substantially parallel with the longitudinal axis.

7. The medical device locator of claim 2, further comprising a controller electrically communicative with the first LED.

8. The medical device locator of claim 7, wherein the controller is configured to actuate the first LED in a pulsating fashion.

9. The medical device of locator claim 8, wherein the controller is in communication with a reciprocating fluid supply, and wherein the controller is configured to substantially synchronize the second inflated volume of the inflatable member with actuation of the first LED and substantially synchronize the first inflated volume of the inflatable member with a deactivation of the first LED.

10. The medical device locator of claim 8, wherein the controller is in communication with the reciprocating fluid supply, and wherein the controller is configured to transition the inflatable member between the first inflated volume and the second inflated volume at a first frequency, and wherein the controller is configurated to actuate the first LED at a second frequency that is larger than the first frequency.

11. The medical device locator of claim 7, further comprising a second LED disposed along the inflatable member and electrically communicative with the controller, wherein the controller is configured to sequentially actuate the first LED and the second LED.

12. The medical device locator of claim 2, wherein the first LED extends helically about the longitudinal axis along a length of the inflatable member.

13. The medical device locator of claim 1, wherein the second inflated volume of the inflatable member has a first diameter larger than a second diameter of a relaxed body lumen.

14. The medical device locator of claim 1, wherein the inflatable member in the first inflated volume is extendable within a body lumen and wherein the inflatable member in the second inflated volume is configured to stretch a wall of the relaxed body lumen such that the wall of the body lumen about the inflatable member is thinner than the wall about the inflatable member in the first inflated volume.

15. A system for locating a medical device, comprising:
a catheter extendable within a first body lumen, the catheter comprising:
an inflatable member disposed about a distal end of the catheter in fluid communication with a fluid lumen extending along the catheter, the inflatable member having a first inflated volume and a second inflated volume larger than the first inflated volume; and
a light emitting diode (LED) disposed along the inflatable member;
an endoscope extendable within a second body lumen toward the catheter between opposing walls of the first and second body lumens.

16. The system of claim 15, further comprising a controller electrically coupled to a fluid supply in fluid communication with the inflatable member, the controller configured to actuate the inflatable member between the first inflated volume and the second inflated volume, and the controller electrically coupled to the LED and configured to substantially synchronize the second inflated volume of the inflatable member with actuation of the LED and substantially synchronize the first inflated volume of the inflatable member with deactivation of the LED.

17. The system of claim 15, further comprising an oscillator coupled to the catheter configured to oscillate the distal end of the catheter across a longitudinal axis when actuated.

18. A method of locating a medical device across a wall of a first body lumen, comprising:
extending an inflatable member comprising a light emitting diode (LED) within the first body lumen;
pulsating the inflatable member between a first inflated volume and a second inflated volume;
actuating the LED during the pulsated second inflated volume; and
using the actuated LED, the second inflated volume of the inflatable member, or both, to locate the medical device from across the wall of the first body lumen.

19. The method of claim 18, further comprising extending a cutting tool from within a second body lumen, across a wall of a the second body lumen toward the wall of the first body lumen and cutting into the wall of the first body lumen.

20. The method of claim 18, wherein using the actuated LED comprises viewing actuated LED from within a space between the wall of the first body lumen and the wall of the second body lumen.
